(19) United States
(12) Patent Application Publication
(10) Pub. No.: US 2023/0045729 A1
(43) Pub. Date: Feb. 9, 2023

(54) IMPLANTABLE LIGHT DELIVERY DEVICE

(71) Applicant: National University of Singapore, Singapore (SG)

(72) Inventors: Daniel Boon Loong TEH, Singapore (SG); Akshaya BANSAL, Singapore (SG); Yong ZHANG, Singapore (SG); Brian Keith KENNEDY, Singapore (SG); Chou CHAI, Singapore (SG); Kah Leong LIM, Singapore (SG)

(21) Appl. No.: 17/904,753

(22) PCT Filed: Mar. 24, 2021
(86) PCT No.: PCT/SG2021/050165

§ 371 Date: Aug. 22, 2022

(30) Foreign Application Priority Data
Mar. 25, 2020 (SG) 10202002770U

(51) Int. Cl. A61N 5/06

(52) U.S. Cl. CPC A61N 5/062; A61N 5/0601; A61N 2005/063; A61N 2005/0659; A61N 2005/0662; A61N 2005/0643

(57) ABSTRACT

An implantable light delivery device comprises: a core portion cladded in a cladding, the core portion comprising upconversion nano-particles (UCNPs) encapsulated in an encapsulation material. The implantable light delivery device may be used in treatments such as photodynamic therapy to provide an emission of light at a wavelength configured by the selection of UCNPs. The encapsulation material may comprise hydrogel and the cladding may comprise fluorinated ethylene propylene.

### TECHNICAL FIELD

The present disclosure relates to implantable light delivery devices for use in photo-based therapies such as photodynamic therapy.

### BACKGROUND

Light delivery into tissue has numerous clinical applications, especially in photodynamic therapy and photo-biomodulation. Photodynamic therapy (PDT) is a clinically approved adjuvant therapy for selective superficial, premalignant cancer and lesion removal. PDT utilizes light excitation to activate a photosensitizer drug, which preferentially accumulates in a tumor, subsequently destroying the tumor with minimal side effects, and excellent cosmetic outcome. However, the low penetration of visible light across tissue limits light-based stimulus therapeutic depth. Furthermore, bulky and tethered light delivery technologies have further prevented its application in deep tissue.

### SUMMARY

According to a first aspect of the present disclosure, an implantable light delivery device comprises: a core portion cladded in a cladding, the core portion comprising up-conversion nano-particles (UCNPs) encapsulated in an encapsulation material.

The present disclosure provides for the use of up-conversion nanotechnology to design a highly flexible optical implant, which is able to transduce deep tissue penetrative invisible light to visible light. The technology can be used directly under enclosed skin to transmit visible light along the implant and even while bending at angles close to 180°, providing tissue depth penetration, flexibility, wireless and battery-free light delivery across tissue. The inventors of the present application have designed the first up-conversion implant that conserves the optical properties of a fiber optics in a hydrogel form.

The core portion and the cladding may be flexible.

The refractive index of the elongate core portion is greater than a refractive index of the cladding. This provides for total internal reflection to take place within the implantable light delivery device.

The encapsulation material may comprise hydrogel.

The core portion may be elongate and the implantable light delivery device may be in the form of an optical fiber.

The cladding may form a tube. One or both ends of the tube may be closed. The cladding may comprise fluorinated ethylene propylene (FEP).

The UCNPs may be selected to absorb near infra-red radiation (700 nm to 1400 nm) and to emit an emission spectrum in the visible range (300 nm to 700 nm) in response to the near infra-red radiation. The emission spectrum overlaps with an absorption spectrum of a photosensitizer.

The concentration of the UCNPs in the core portion is substantially uniform along the core portion. This provides for uniform emission from the implantable light delivery device.

The concentration of the UCNPs in the core portion is varies along the core portion. This provides for a concentration of emission at parts of the device where the concentration of UCNPs is higher.

The implantable light delivery device may be formed from a bundle of fibers which can be placed around a tumor. Thus, allowing for illumination to be provided to an irregularly shaped tumor.

The implantable light delivery device may be provided with a portion configured as a deformable cup or a hollow sphere. Such a portion can be placed in an irregular cavity left following resection of a tumor. The implantable light delivery device may be provided with a portion formed as a solid sphere.

The UCNPs may be configured to emit a first visible light emission in response to a first near infra-red radiation excitation and to emit a second visible light emission in response to a second near infra-red radiation excitation.

According to a second aspect of the present disclosure, a photodynamic therapy method is provided. The method comprises implanting an implantable light delivery device into a subject; administering a photosensitizer to the subject; and illuminating the implantable light delivery device with radiation having excitation spectra corresponding to the UCNPs.

According to a third aspect of the present disclosure a method of manufacturing an implantable light delivery device is provided. The method comprises: molding a mixture of an encapsulation material and up-conversion nano-particles (UCNPs); and causing the encapsulation material to polymerize.

The mixture may be molded in a cladding. The encapsulation material may be caused to polymerize by irradiating the encapsulation material with UV radiation.

### BRIEF DESCRIPTION OF THE DRAWINGS

In the following, embodiments of the present invention will be described as non-limiting examples with reference to the accompanying drawings in which:

FIG. 1A and FIG. 1B show an implantable light delivery device according to an embodiment of the present invention;

FIG. 2A to FIG. 2C show steps in a method of manufacturing implantable light delivery devices according to embodiments of the present invention:

FIG. 3A and FIG. 3B illustrate the use of an implantable light delivery device according to an embodiment of the present invention in treatment of a subject;

FIG. 4A to FIG. 4C show variations that can be made to the design of the implantable light delivery device in embodiments of the present invention;

FIG. 5A to FIG. 5C show end configurations of implantable light delivery devices according to embodiments of the present invention;

FIG. 6A to FIG. 6C show a cup shaped implantable light delivery device according to an embodiment of the present invention;

FIG. 7A and FIG. 7B show an implantable light delivery device configured to emit different wavelengths of visible light in response to different excitation NIR radiation;

FIG. 8 shows an example implementation of an implantable light delivery device according to an embodiment in a mice glioblastoma model;

FIG. 9A shows the absorption spectra of the photosensitizer protoporphyrin (PpIX);

FIG. 9B shows the emission spectra from a mixture of UCNPs used in embodiments of the present invention;

FIG. 10A shows a shows direct visualization of Er and Tm based UCNPs and their resultant mixture emission in solution;

FIG. 10B shows an image of an Er and Tm doped PEGDA based UCNP implantable light delivery device;

FIG. 11A to FIG. 11D show the effect on emission of bending an implantable light delivery device according to an embodiment of the present invention;

FIG. 12A and FIG. 12B are images of an implantable light delivery device according to embodiments of the present invention showing the penetration of emission;

FIG. 13A and FIG. 13B show optical and physical characterization of the implantable light delivery devices according to embodiments of the present invention;

FIG. 14A to FIG. 14E show a demonstration of photodynamic theory (PDT) using implantable light delivery devices according to embodiments of the present invention;

FIG. 15 shows a set of in-vivo images showing the impact on tumors of PDT treatment using an implantable light delivery device according to an embodiment of the present invention;

FIG. 16 shows results of an experiment to demonstrate PDT using an implantable light delivery device according to an embodiment of the present invention;

FIG. 17 illustrates an application of an implantable light delivery device according to an embodiment of the present invention under the skin;

FIG. 18A and FIG. 18B show scalp temperatures of a mouse when undergoing PDT treatment using an implantable light delivery device according to an embodiment of the present invention; and

FIG. 19A and FIG. 19B illustrate the biocompatibility of an implantable light delivery device according to an embodiment of the present invention.

### DETAILED DESCRIPTION

The present disclosure provides an up-conversion optical implant or implantable light delivery device in different shapes and sizes, which may be referred to as an up-conversion nano-particle (UCNP) optrode. The idea for an up-conversion implant stems from the need to deliver visible light deep into tissue for an array of photo-based therapies including photodynamic therapy (PDT) and photo-biomodulation. Visible light has poor tissue penetration ability as compared to longer wavelengths near-infrared (NIR). However, as most biological moieties do not respond to NIR, a transducer for NIR to visible is needed. This is the first of its kind up-conversion hydrogel-based implant for wireless visible light emission.

Up-conversion nano-particles (UCNPs) are a unique class of fluorescent nanoparticles that convert low energy NIR to high energy (300 nm to 700 nm) visible/UV light in a non-linear optical process known as up-conversion. In doing so, they serve as nano-transducers for phototherapy applications, that would benefit from the use of deeper penetrating NIR light, while relying on light sensitive targeted moieties that cannot be directly activated by NIR. UCNPs are tunable in their emission, shape and size through rational design and dopant type selection in addition to having desirable optical properties such as absence of photo-bleaching and photo-blinking (2). However, the direct introduction of UCNP suspension into tissues, poses several issues such as diffusion and retention of UCNPs in the central nervous system, thereby limiting its translational potential in actual clinical setting.

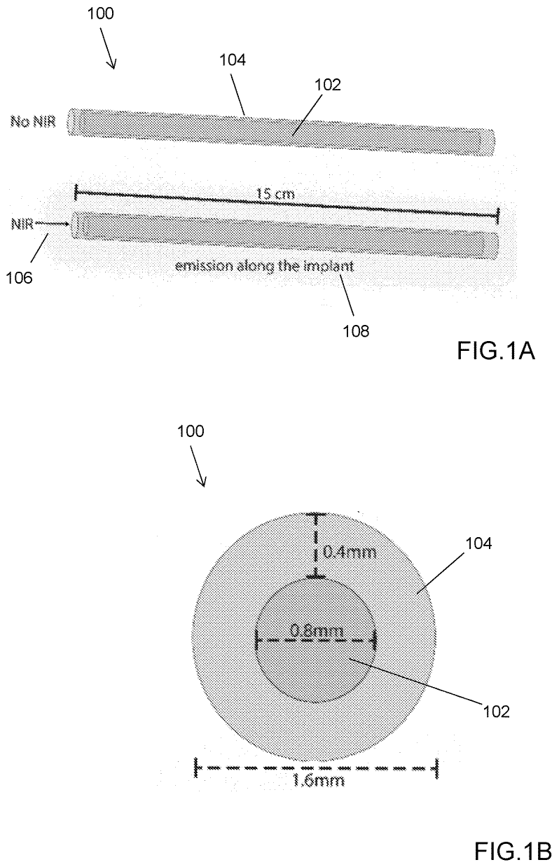

FIG. 1A and FIG. 1B show an implantable light delivery device according to an embodiment of the present invention. FIG. 1A is a side on view and FIG. 1B is a cross sectional view. As shown in FIG. 1A, the implantable light delivery device 100 or UCNP hydrogel optical fiber, is an elongate and has the form of an optical fiber. The implantable light delivery device 100 comprises a core portion 102 formed from UCNPs encapsulated in a hydrogel and a cladding 104 formed from fluorinated ethylene propylene (FEP). When the implantable light delivery device 100 is illuminated with NIR 106 at one end, this NIR excitation results in in visible light emission along the entire implantable light delivery device 100. The implantable light delivery device 100 acts as an optical fiber to guide the excitation NIR radiation and the UCNPs convert the excitation NIR radiation into visible light emission. As shown in FIG. 1A, the implantable light delivery device has a length of 15 cm. As shown in FIG. 1B, the implantable light delivery device is circular in cross section and the core portion 102 has a diameter of 0.8 mm, the cladding 104 has a thickness of 0.4 mm and the overall thickness of the implantable light delivery device is 1.6 mm. The cladding 104 is formed as an FEP tube for cladding, which functions as a mold when the implantable light delivery device 100 is manufactured and also functions to provide internal optical reflection when the implantable light delivery device 100 is in use.

It will be appreciated that the dimensions described above are intended as an illustrative example and the dimensions of the implantable light delivery device can be adjusted in accordance with clinical needs to fit within a human or animal subject.

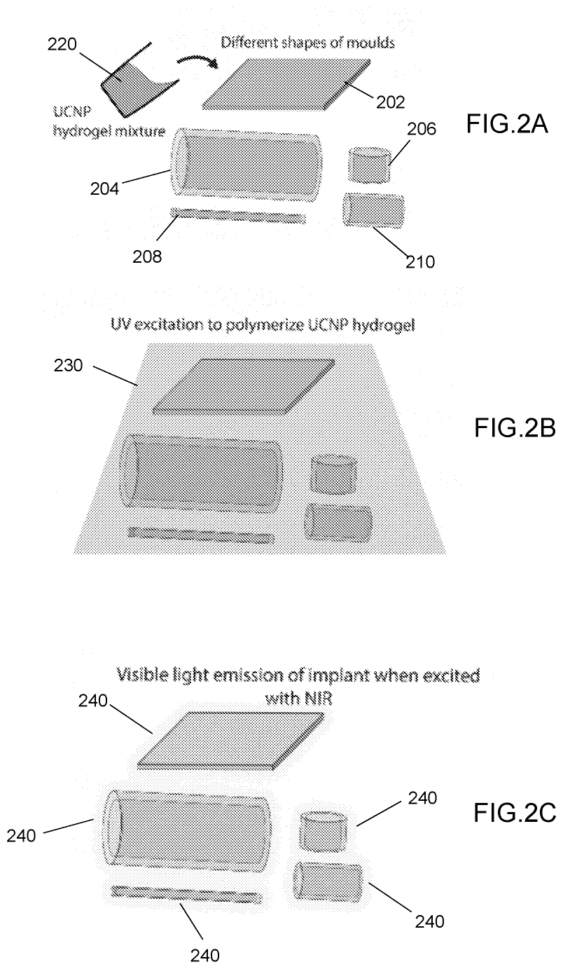

FIG. 2A to FIG. 2C show steps in a method of manufacturing implantable light delivery devices according to embodiments of the present invention. As shown in FIG. 2A, different shapes of mold may be used. Molds having a planar shape 202, a wide cylindrical shape 204, a short cylindrical shape, a fiber shape 208 and small cylindrical shape 210 are formed from FEP which will form the cladding of the implantable light delivery device. The molds are filled with a mixture of UCNPs and hydrogel 220. The hydrogel may be Poly(ethylene glycol) diacrylate (PEGDA).

As shown in FIG. 2B, the UCNP and hydrogel filled molds are subjected to ultraviolet (UV excitation) 230 to polymerize the hydrogel into a flexible solid.

As shown in FIG. 2C the implantable light delivery devices are forms and excitation with NIR can achieve visible light emission 240 across the implant, depending on the size of NIR excitation and strength. As the shapes are derived from the mold, the range of possible sizes here such as the length can be 5 cm to 20 cm, the diameter of the core can be 0.5 mm to 5 mm, the diameter of the implant can be 1 mm to 5 mm.

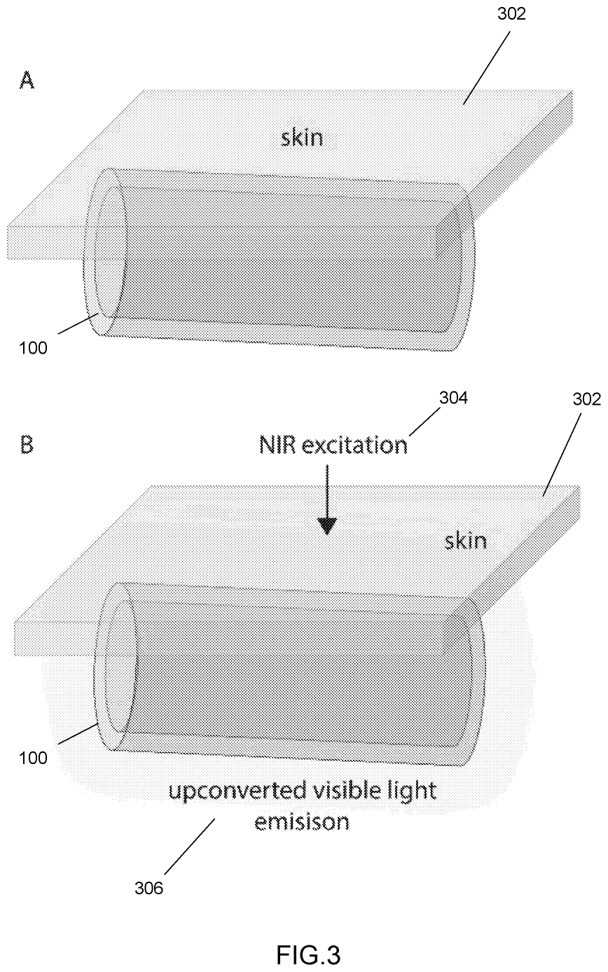

FIG. 3A and FIG. 3B illustrate the use of an implantable light delivery device according to an embodiment of the present invention in treatment of a subject. The subject may be a human patient or animal.

As shown in FIG. 3A, the implantable light delivery device 100 is implanted under the skin 302 of the subject. The implantable light delivery device can be implanted underneath tissue with the skin 302 sutured closed.

As shown in FIG. 3B, NIR excitation 304 is applied above the skin 302. The NIR excitation 304 excites the UCNPs in the implantable light delivery device and causes emission of upconverted visible light 306 underneath the skin 302 of the subject.

In some embodiments, the implantable light delivery device may be used in a photodynamic therapy treatment method. In such a method, a photosensitizer drug is administered to the subject and the upconverted visible light emission under the skin of the patient activates the photosensitizer drug.

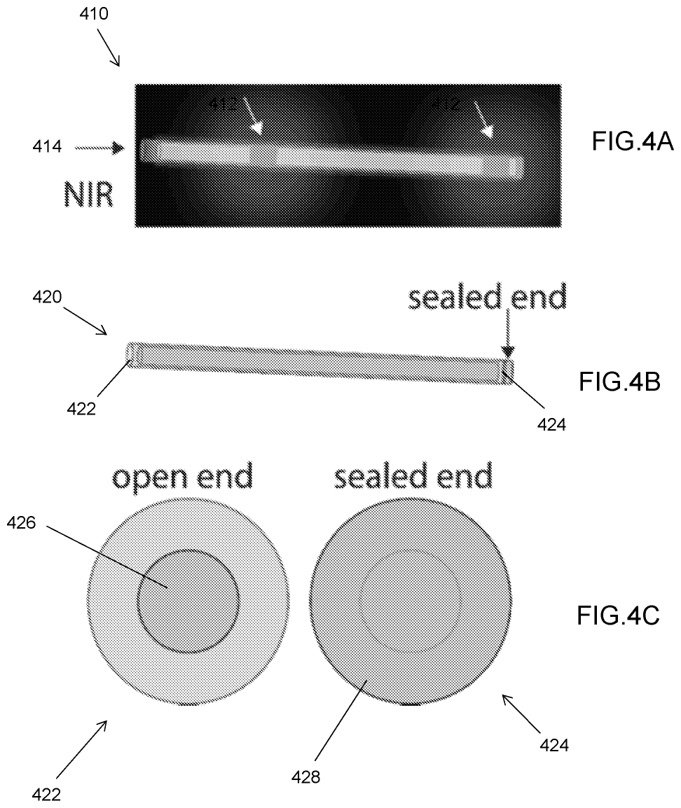

FIG. 4A to FIG. 4C show variations that can be made to the design of the implantable light delivery device in embodiments of the present invention. As shown in FIG. 4A, in an implantable light delivery device 410, localized emission can be controlled by tuning the UCNP concentrations at points of interest within the implantable light delivery device 410. Increased concentrations of UCNPs are provided at two locations 412. With the implantable light delivery device 410 is subjected to NIR excitation emission 414, the visible light emission is concentrated at the two locations 412.

As shown in FIG. 4B and FIG. 4C, an implantable light delivery device 420 has an open end 422 and a sealed end 424. FIG. 4C shows a cross section of the open end 422 and the sealed end 424. The open end 422 has an opening 426 which allows the UCNPs and hydrogel to be inserted. The sealed end 424 is covered by a layer of FEP. In some embodiments, the open end can be sealed by low heating of the FEP tube. This sealing of the open end may take place after the UCNPs and hydrogel have been inserted.

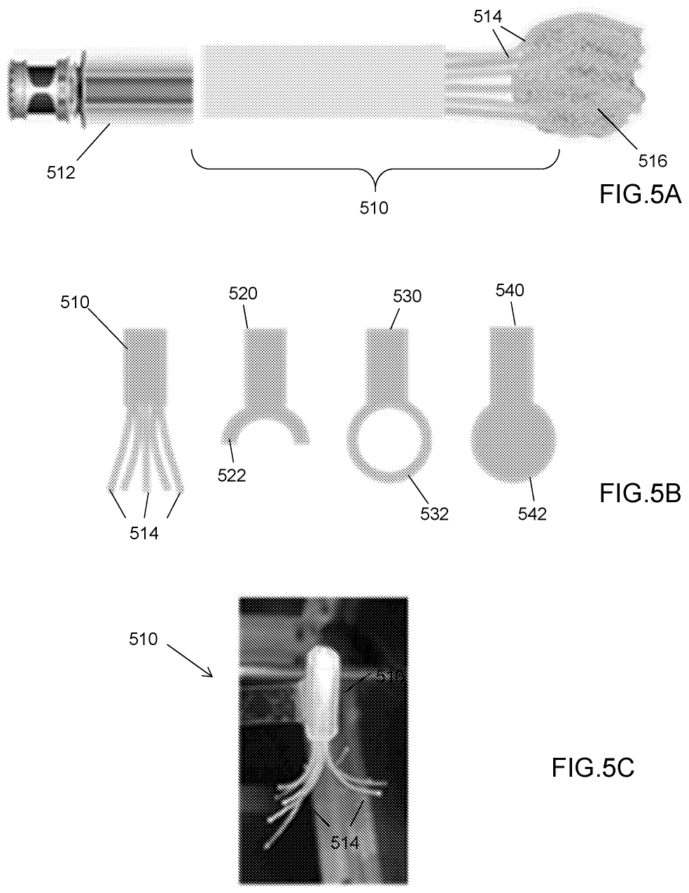

FIG. 5A to FIG. 5C show end configurations of implantable light delivery devices according to embodiments of the present invention. In some embodiments an end of the implantable light delivery device may be configured to target a tumor or cavity. It is noted that post resection, a tumor cavity may be irregular and the shape of the tumor cavity will depend on the nature of the tumor. Additionally, if the implantable light delivery device is used to treat a tumor, the implantable light delivery device must be configurable to effectively illuminate the tumor, for example to effectively activate a photosensitizer drug for treatment of the tumor.

FIG. 5A shows an implantable light delivery device in use to illuminate a tumor with visible light. As shown in FIG. 5A, the implantable light delivery device 510 implanted below the skin of the subject and is illuminated by an NIR laser 512 above the skin of the subject. An end of the implantable light delivery device 510 distal to the NIR laser 512 comprises a bundle of elongate fiber portions 514 which placed around a target tumor 516.

FIG. 5B shows a variety of possible tips shapes for implantable light delivery devices according to embodiments of the present invention. As shown in FIG. 5B, one design for an implantable light delivery device 510 comprises a plurality of fiber portion 514 extending from one end. A second design for an implantable light delivery device 520 comprises a cup portion 522 on one end of the device. A third design for an implantable light delivery device 530 comprises a hollow sphere 532 provided on one end. A fourth design for an implantable light delivery device 540 comprises a sold sphere provided on one end. The second to fourth designs for implantable light delivery devices 520-540 may be provided in a large cavity post resection. The cup portion 522 or the hollow sphere 532 may be deformed to fit an irregular cavity.

FIG. 5C shows a photograph of an implantable light delivery device comprising a plurality of fibers according to an embodiment of the present invention. As shown in FIG. 5C, the implantable light delivery device 510 comprises a plurality of fibers 514 which extend inside the main body 516 of the implantable light delivery device 510

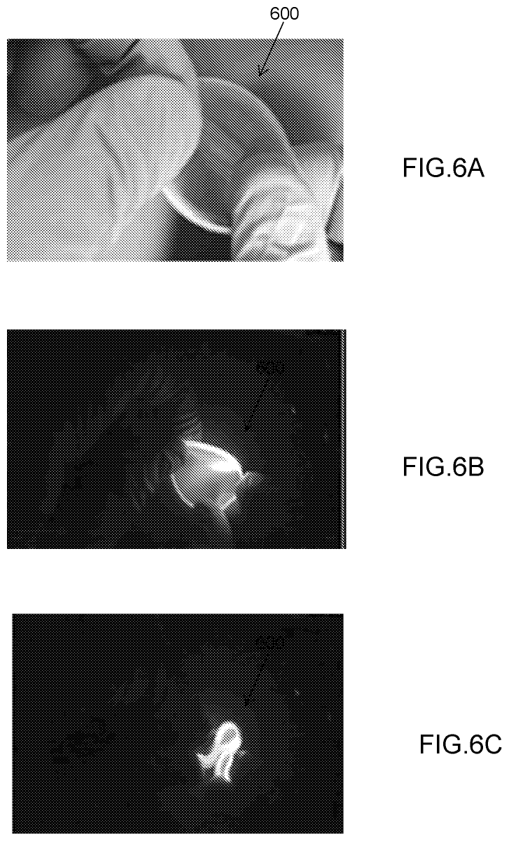

FIG. 6A to FIG. 6C show a cup shaped implantable light delivery device according to an embodiment of the present invention. As shown in FIG. 6A, the implantable light delivery device 600 is flexible. The design shown in FIG. 6A to FIG. 6C is based on Polydimethylsiloxane (PDMS). FIG. 6B and FIG. 6C show the implantable light delivery device when illuminated. FIG. 6B shows the implantable light delivery device in its normal shape and FIG. 6C shows the implantable light delivery device when flexed, for example to fit the device into a cavity. As can be seen from FIG. 6C, even when the implantable light delivery device 600 is flexed or crumpled, it still functions to provide illumination.

In some embodiments, the UCNPs are configured to emit different wavelengths of visible light depending upon the NIR excitation wavelength. As described in Mei, Q., Bansal, A., Jayakumar, M. K. G. et al. Manipulating energy migration within single lanthanide activator for switchable upconversion emissions towards bidirectional photoactivation. Nat Commun 10, 4416 (2019), the cross-relaxation (CR), a non-radiative recombination pathway typically perceived as deleterious, to manipulate energy migration within the same lanthanide activator ion (Er³⁺) towards orthogonal red and green emissions, simply by excitation wavelength from 980 to 808 nm. These UCNPs allow programmable activation of two synergistic light-gated ion channels VChR1 and Jaws in the same cell to manipulate membrane polarization, demonstrated for cardiac pacing.

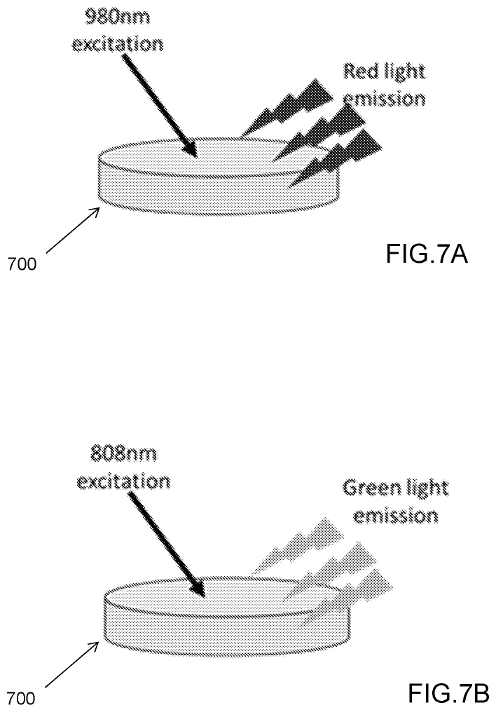

FIG. 7A and FIG. 7B show an implantable light delivery device configured to emit different wavelengths of visible light in response to different excitation NIR radiation. The implantable light delivery device 700 is provided with UCNPs having the luminescence activator Er³⁺ incorporated into the core structure. As shown in FIG. 7A, when the implantable light delivery device 700 is illuminated with NIR excitation radiation having a wavelength of 980 nm, red light (having a wavelength of approximately 650 nm) emission occurs. As shown in FIG. 7B, when the implantable light delivery device 700 is illuminated with NIR excitation radiation having a wavelength of 808 nm, green light (having a wavelength of approximately 540 nm) emission occurs.

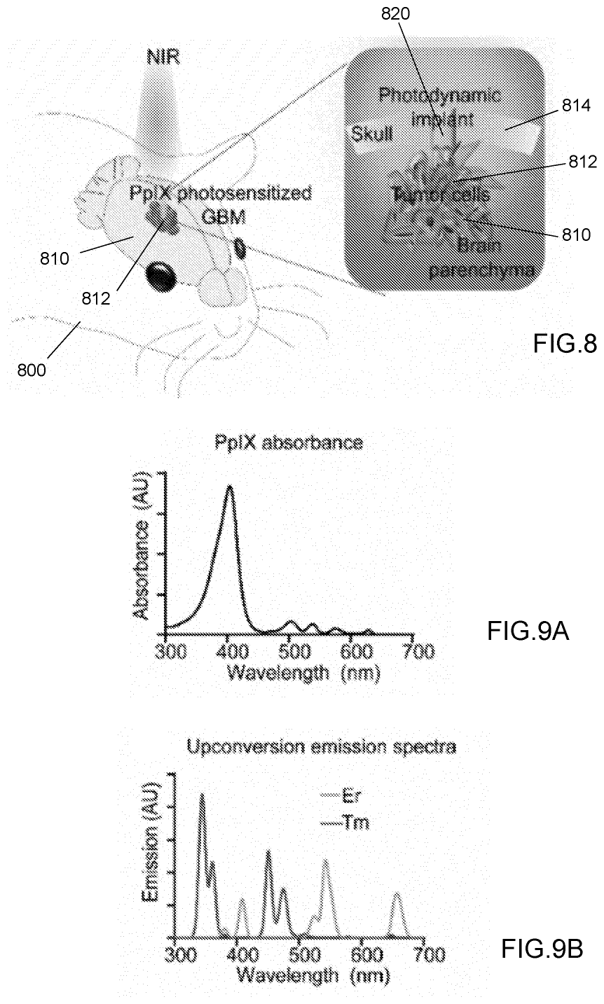

An example implementation of an implantable light delivery device according to an embodiment will now be described with reference to FIG. 8 to FIG. 12B. The concept of deep-penetrating, wireless, flexible and electrical free light delivery for PDT is demonstrated for mice glioblastoma (GBM) model.

FIG. 8 shows an example implementation of an implantable light delivery device according to an embodiment in a mice glioblastoma model. As shown in FIG. 8, implantable light delivery device 820 was implanted in the brain 810 of a mouse 800. The implantable light delivery device 820 was implanted close to a tumor 812 through an incision in the skull 814 of the mouse 800. The photosensitizer protoporphyrin (PpIX) was administered and the implantable light delivery device 820 was illuminated with NIR excitation radiation.

FIG. 9A shows the absorption spectra of the photosensitizer protoporphyrin (PpIX). As can be seen from FIG. 9A there is a peak in the absorption spectra at a wavelength of around 400 nm.

FIG. 9B shows the emission spectra from a mixture of NaYF4:Yb/Er@SiO2 and NaYF4:Yb/Tm@SiO2 UCNPs. Comparing FIG. 9A and FIG. 9B, it can be seen that the combination of NaYF4:Yb/Er@SiO2 and NaYF4:Yb/Tm@SiO2 UCNPs provides a close correspondence to the absorption spectra of PpIX.

For photodynamic therapy, a common photosensitizer, PpIX's absorption spreads across the visible spectrum peaking at the Soret band of 405 nm with several smaller longer wavelength peaks, termed the Q band as shown in FIG. 9A. Excitation of PpIX with 405 nm, the absorption maxima, generates a single emission peak at 635 nm, which is useful for fluorescence-based quantification of PpIX. The efficacy of PDT excitation on PpIX should optimally be adjusted to the Soret band absorption, but this is also the lower tissue penetrative wavelength as compared to the Q band's absorption. UCNPs can be tuned to emit across the UV and visible range of light by careful selection of lanthanide ion dopants in an inorganic sodium rare earth metal halide matrix, most commonly, NaYF4. This matrix is chosen for its low phonon energy and high upconversion efficiency. The implant was tuned to match the absorption spectrum of PpIX by using two types of UCNPs, NaYF4:Yb/Er@SiO2 to emit blue, green and red light, while using NaYF4:Yb/Tm@SiO2 to emit UV and blue light as shown in FIG. 9B when excited with NIR (980 nm). This achieves the dual objectives of both deep tissue penetration and PDT efficacy.

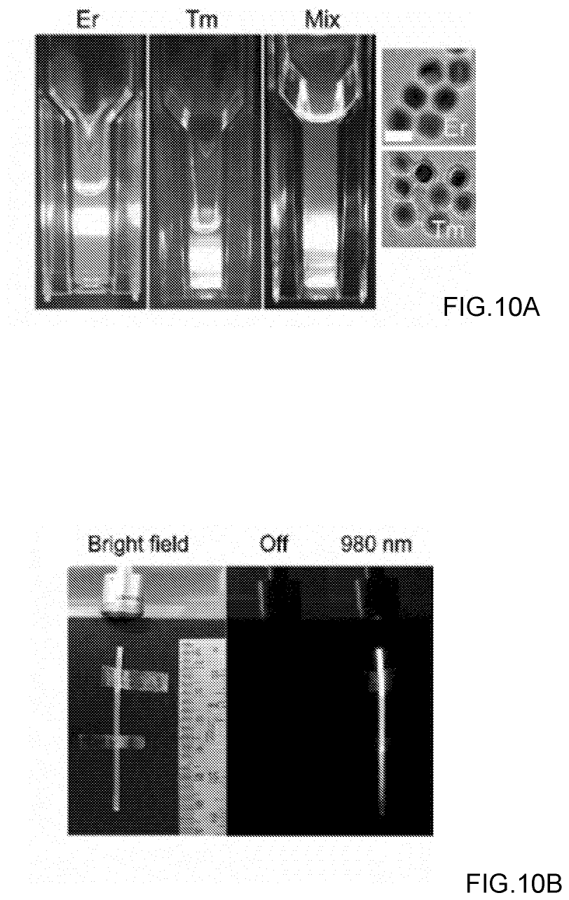

FIG. 10A shows a shows direct visualization of Er and Tm based UCNPs and their resultant mixture emission in solution. The images were captured with a transmission electron microscope (TEM). The scale bar indicates 50 nm. As can be seen from FIG. 10A, the UCNPs have a diameter of approximately 50 nm.

FIG. 10B shows an image of an Er and Tm doped PEGDA based UCNP implantable light delivery device. As can be seen from FIG. 10B, when illuminated with excitation NIR radiation at 980 nm, visible light is emitted by the full length of the implantable light delivery device.

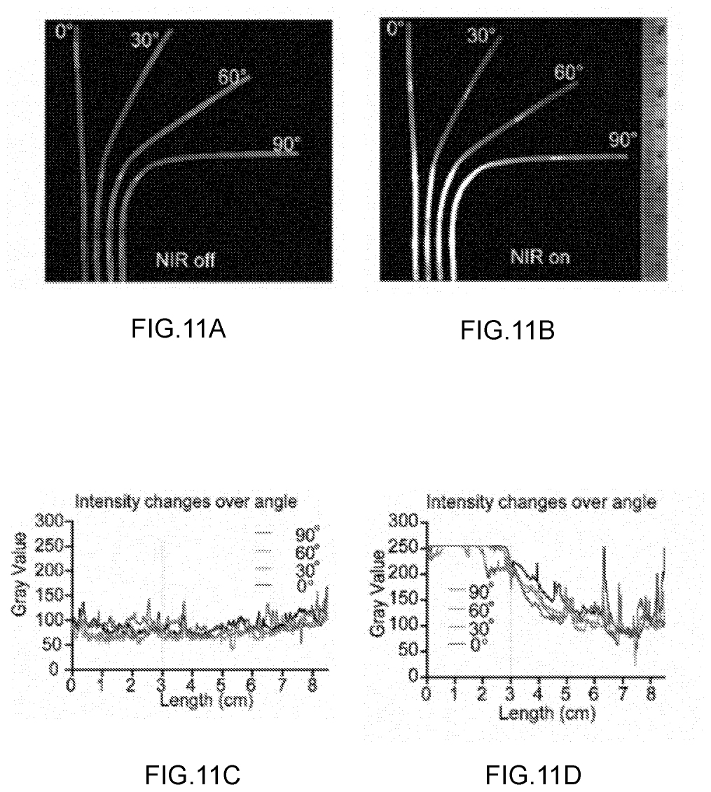

FIG. 11A to FIG. 11D show the effect on emission of bending an implantable light delivery device according to an embodiment of the present invention. FIG. 11A is an image of the implantable light delivery device without NIR excitation with bending angles of 0 degrees, 30 degrees, 60 degrees and 90 degrees. FIG. 11B is an image of the implantable light delivery device with NIR excitation at 1583 mW/cm² with bending angles of 0 degrees, 30 degrees, 60 degrees and 90 degrees.

FIG. 11C shows the intensity change over angle for the implantable light delivery device without NIR excitation with bending angles of 0 degrees, 30 degrees, 60 degrees and 90 degrees. FIG. 11D shows the intensity change over angle for the implantable light delivery device with NIR excitation with bending angles of 0 degrees, 30 degrees, 60 degrees and 90 degrees. The bending took place at 3 cm. As can be seen from FIG. 11D, there is a decrease in emission caused by the bending however, even after 6-7 cm there is still a noticeable increase above the gray values shown in FIG. 11C.

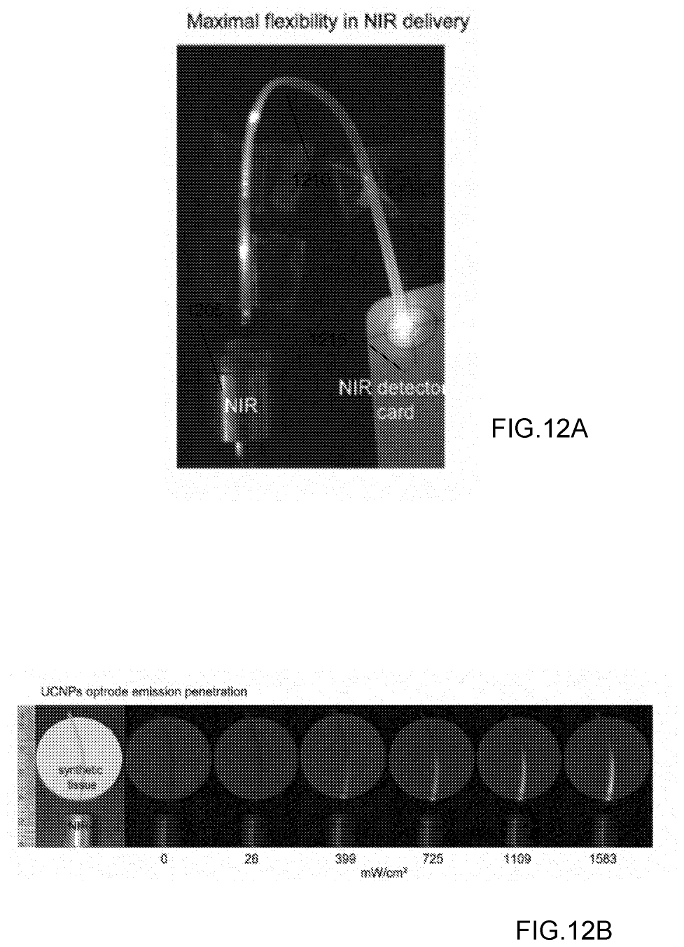

FIG. 12A and FIG. 12B are images of an implantable light delivery device according to embodiments of the present invention showing the penetration of emission. FIG. 12A shows emission by an NIR laser into an implantable light delivery device 1210 and the detection of emission by the implantable light delivery device 1210 by an NIR detector card 1215. As shown in FIG. 12A, even when the implantable light delivery device 1210 is bend through an angle of 180 degrees, there is still an emission onto the NIR detector card 1215.

FIG. 12B shows visible light emission when the implantable light delivery device is implanted into synthetic tissue for different excitation NIR powers. As shown in FIG. 12B, for excitation powers of 725 mW/cm², 1109 mW/cm² and 1583 mW/cm² the visible emission along the implantable light delivery device can clearly be seen.

For flexibility and biocompatibility, the implantable light delivery devices are fabricated with a Polyethylene (glycol) Diacrylate (PEGDA) core cladded with FDA approved fluorinated ethylene propylene (FEP). The implantable light delivery devices are specifically designed to allow total internal reflection such that the implant could provide light guiding functionality based on PEGDA hydrogel light guiding properties. Fibers constructed from 10% wt/v PEGDA have a refractive index close to 1.35, while these would be light guiding in the air (n=1), the situation varies in tissues. To ensure our implant can serve as a light guide in any medium, FEP with a refractive index of 1.344 was added as cladding. The core consisted of a 10% wt/v PEGDA hydrogel encapsulating the two types of silica coated UCNPs in a 1:1 ratio. The refractive index of the core is >1.35 (n_{water}=1.33, nUCNP˜1.6,2 n_{PEGDA}˜1.47), meeting the conditions for total internal reflection to occur, which is that the refractive index of the core portion is greater than the refractive index of the cladding. NIR to visible light spectra transduction with UCNPs implant was achieved tethered-free even when embedded in synthetic tissue, thus enabling remote activation of the implant in deep tissue. Flexible light guiding capacity of UCNPs implant can be conserved along the implant even upon bending at different angles. One notable feature of the implant, not previously reported, is the transmission of NIR across the PEGDA even when bent at maximal angle—see FIG. 12A.

Emission intensity relies on several factors: i) the power density of NIR and ii) concentration of the UCNP in the hydrogel. To be clinically applicable, the implantable light delivery devices have been designed to work below the human permissible limit to NIR excitation at 726 mW/cm², when implanted underneath the skin or tissue. Localized high intensity emission can be achieved implantable light delivery devices by selectively increasing localized concentration of UCNP. This is done during the polymerization process, by virtue of selectively pouring concentrate UCNP hydrogel liquid to desired area of high intensity emission. Once UV excited, the implant will polymerize and solidifies. In contrast, uniform distribution of UCNP hydrogel mixture can be achieved by constant rotation of the optrode during UV excitation for polymerization.

Furthermore, in the case of NIR, the transmission extended even to 180° of bending, indicating that the PEGDA-UCNPs hybrid may be a favorable NIR optical fiber solution. Additionally, with the FEP cladding, UCNPs leaching from the implant can be reduced to minimal. Besides hydrogel, the implantable light deliver devices can also be made with Polydimethylsiloxane (PDMS). However, PDMS will not possess the optical guiding properties of that to hydrogel.

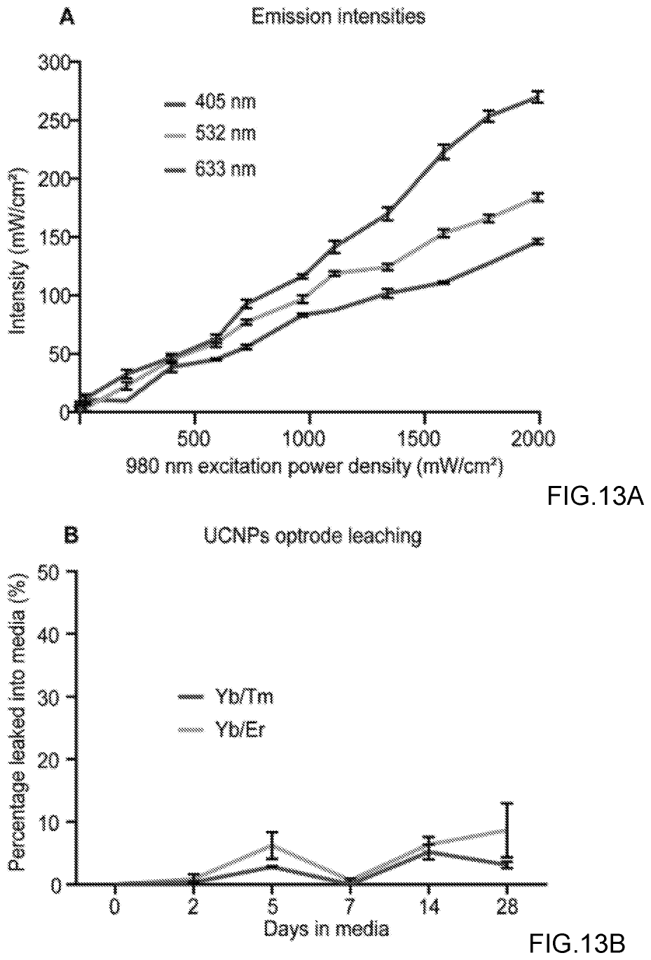

FIG. 13A and FIG. 13B show optical and physical characterization of the implantable light delivery devices. FIG. 13A shows emission intensities from a 5 cm implantable light delivery device when excited with NIR at increasing intensities. FIG. 13B shows UCNPs leaching percentage in cell culture media DMEM (Dulbecco's modified Eagle's medium) from an implantable light delivery device, over 28 days. Minimal leaching not exceeding 8.6±4% was observed for an implant continuously submerged in DMEM (n=3 experiments).

the implantable light delivery device can be implanted into tissue and the skin will be sutured closed for non-invasive activation of the implant. Wireless NIR excitation can then be performed across the skin with 980 nm excitation to activate the implant which will emit visible light to target photosensitizer.

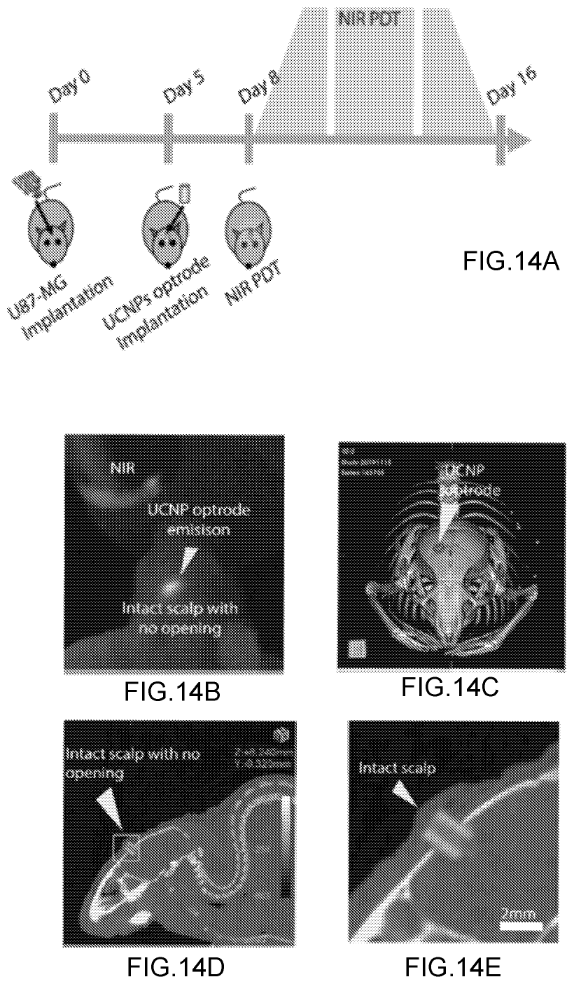

FIG. 14A to FIG. 14E show a demonstration of photodynamic theory (PDT) using implantable light delivery devices according to embodiments of the present invention. FIG. 14 A illustrates the in-vivo NIR PDT experiment schedule with an implantable light delivery device incorporating UCNPs. As illustrated in FIG. 14A, on day 0, a U87-MG tumor was implanted in the mouse brain. On day 5, the implantable light delivery device was implanted. Photodynamic theory (PDT) was carried out form day 8 to day 16 by illuminating the implanted light delivery device with NIR excitation radiation.

FIG. 14B shows an image of the emission from the implantable light delivery device. AS shown in FIG. 14B, the visible emission from the implantable light delivery device incorporating UCNPs can be visualized when excited with 725 mW/cm2 of NIR.

FIG. 14C is a microCT scan showing the implant location and depth of penetration. AS shown in FIG. 14C, the implantable light delivery device was secured to the skull with surgical glue.

FIG. 14D and FIG. 14E are microCT scan showing the implantable light delivery device incorporating UCNPs secured on the skull. For wireless NIR-PDT in vivo, the implantable light delivery device incorporating UCNPs can be readily secured on the skull or skin with only surgical glue. As shown in FIG. 14D and FIG. 14E, the scalp was sutured closed entirely and left to be healed. Implant emission could be directly visualized through the closed and healed scalp, when excited with 725 mW/cm² of NIR (980 nm). Notably, this power density is within the conservative limits for human skin exposure to 980 nm excitation (726 mW/cm²). Due to the flexibility of hydrogel and FEP cladding, mobility of the organ is not affected.

FIG. 14 shows a set of in-vivo images showing the impact on tumors of PDT treatment using an implantable light delivery device according to an embodiment of the present invention. The untreated images show development of the tumor when no treatment was applied. The 5-ALA images show development of the tumor when 5-aminolevulinic acid which is a photosensitizer was administered but no illumination was applied. The NIR images show the development of the tumor when NIR illumination was applied with no implantable light delivery device present. Finally, the PDT images show the development of the tumor when the implantable light delivery device was present.

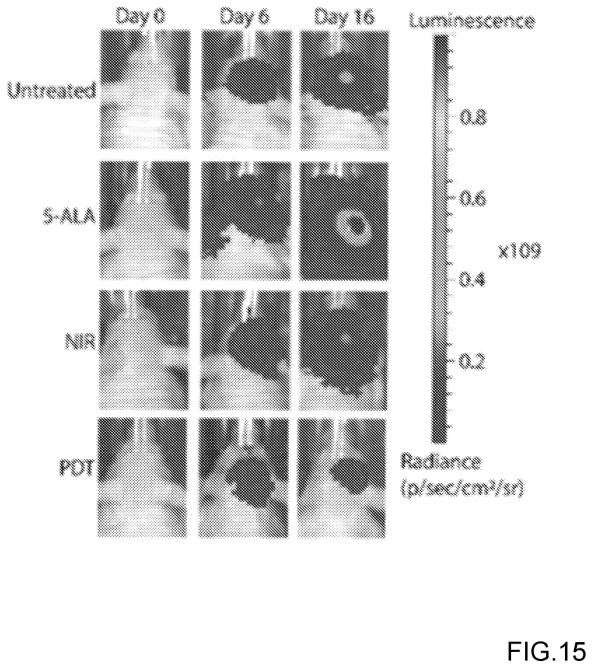

As can be seen from FIG. 15, the tumor was present and approximately the same size in each case on day 6 (which was before the PCT was commenced). The application of PDT with the implantable light delivery device reduced the size of the tumor whereas in all of the other cases, the size of the tumor increased. Thus the images indicate PDT mouse tumors were regressing as compared to other controls groups.

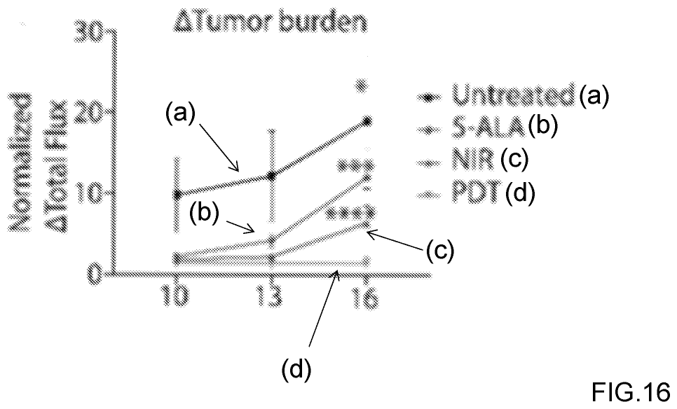

FIG. 16 shows results of the experiment to demonstrate PDT using an implantable light delivery device according to an embodiment of the present invention. FIG. 16 shows the shows the normalized change of tumor burden in all experiment groups over time. *p=0.0327, ***p=0.0002, ****p<0.0001.

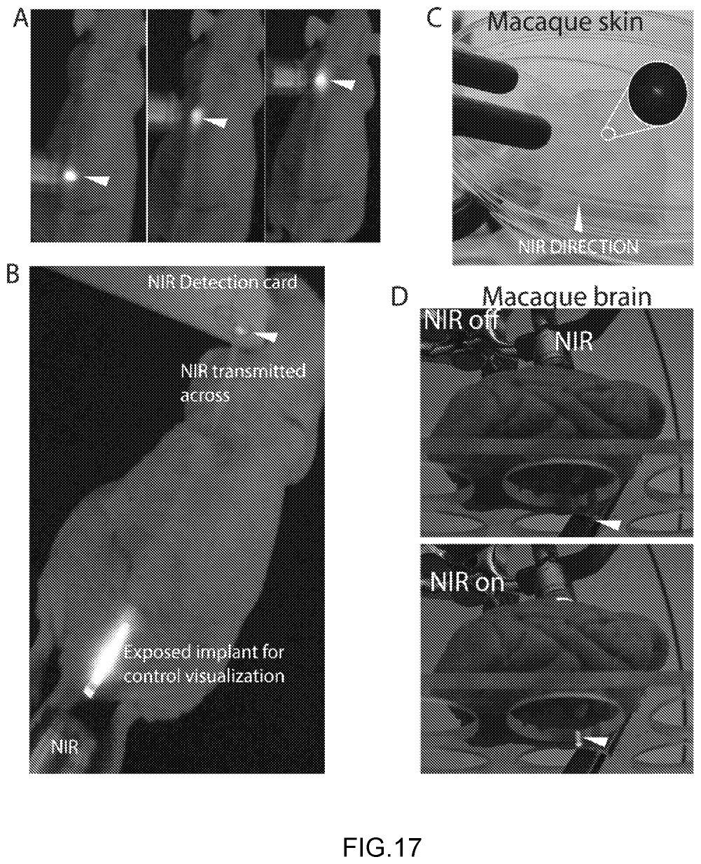

FIG. 17 illustrates an application of an implantable light delivery device according to an embodiment of the present invention under the skin. FIG. 17A shows an evaluation of potential subcutaneous applications of the technology by inserting the implant underneath the skin along the back of the mice. FIG. 7A shows NIR delivery across UCNPs implant from the excitation point, proximal to the tail of the mouse, till the neck of the mouse. As shown in FIG. 17A, when irradiated transdermally with NIR, excitation of the implant along the back of the mice could be clearly observed.

FIG. 17B shows NIR transmission through an implantable light delivery device according to an embodiment of the present invention. As shown in FIG. 17B, the NIR transmission property was retained in the implant, even when placed underneath the skin. The arrow points to the emission from the implant when transdermally activated with NIR.

FIG. 17C and FIG. 17D shows implantation of an implant under macaque skin and in a macaque brain. FIG. 17C show activation of the UCNPs in the implant was also achieved across macaque skin. FIG. 17C shows wireless visible light emission was achieved even transdermally across macaque skin. UCNPs implant was placed in the dermis region, while NIR was excited across the epidermis region, as indicated by the white arrow. These findings demonstrated the feasibility of using the UCNPs-based implant for light-based therapeutics beyond the brains.

As shown in FIG. 17D, the implantable light delivery device when inserted through a fixed macaque brain, is able to emit visible light across the brain. The tip of the implant is shown with the white arrow. Upconverted visible light can be seen emitted from the ventral tip of the implant when excited with NIR

To demonstrate the scalability of the waveguiding UCNPs optrode technology for deeper light delivery, for e.g. for human applications, we showed the feasibility of our system in larger brain, that of a cynomolgus macaque (Macaca fascicularis). To do this, an UCNP implant was inserted from the dorsal surface extending through to the ventral part of a fixed macaque brain. Following NIR (725 mW/cm²) excitation at the dorsal tip of the implant, visible emission was observable at the ventral tip FIG. 17D, implying that UCNP implant is able to transmit light up across brain tissue in which it was embedded.

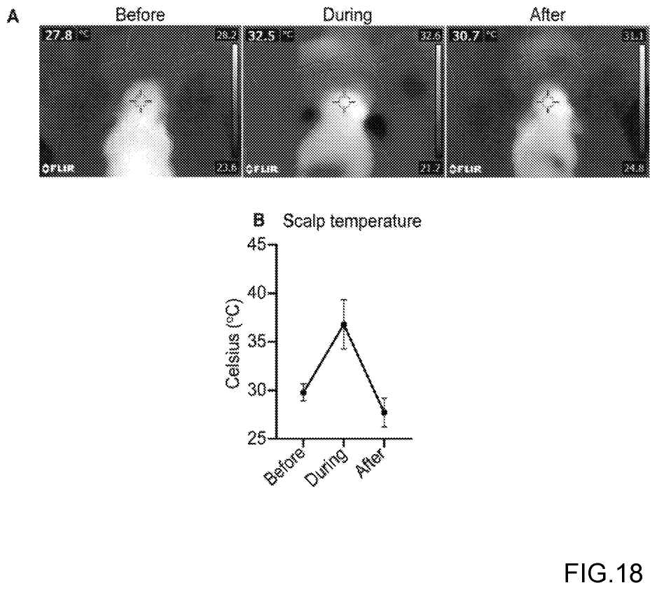

FIG. 18A and FIG. 18B show scalp temperatures of a mouse when undergoing PDT treatment using an implantable light delivery device according to an embodiment of the present invention. FIG. 18A shows the scalp temperature of mouse before during and after NIR-PDT. FIG. 18B shows the average scalp temperatures for mice before, during and after NIR PDT.

As shown in FIG. 18A and FIG. 18B, with proper intermittent application of localized icepack cooling, the scalp temperature was maintained at manageable physiological range during NIR. However, improvement in scalp temperature can also be performed with 800 nm based UCNPs optrode.

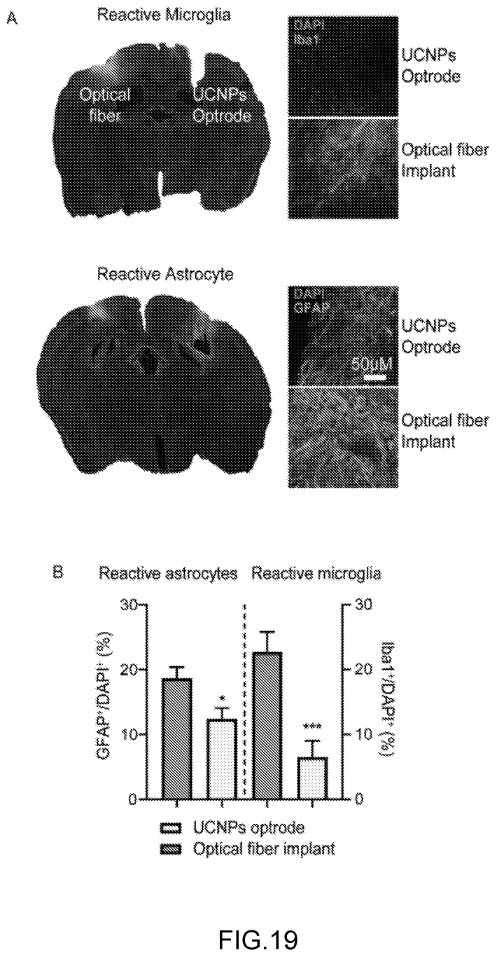

FIG. 19A and FIG. 19B illustrate the biocompatibility of an implantable light delivery device according to an embodiment of the present invention. FIG. 19A shows Reactive microglia (Iba1+) and reactive astrocyte (GFAP+) immunohistochemistry on mice brain implanted with fiber optic on the left, and the implantable light delivery device incorporating UCNPs (UCNPs optrode) on the right. More intense signal can be detected on the fiber optic hemisphere, which suggests that the implantable light delivery device cladded in FDA approved FEP has better biocompatibility and less glia reactivity. Implant was implanted for 2 weeks. FIG. 19B shows the quantification of reactive astrocyte (GFAP+/DAPI+) and reactive microglia (Iba1+/DAPI+) normalized fraction respectively. In both reactive astrocytes and reactive microglias, UCNPs optrode have significantly lower percentage of positive fraction. *p=0.0259, ***p=0.0008.

FIG. 19A and FIG. 19B show the biocompatibility of the I implantable light delivery device in the brains of normal immunocompetent C57BL/6 mice in comparison with implantable optical fiber. FEP is bio-inert and has been used extensively in medical or animal implants. We performed concurrent bilateral comparison of glial reactivity between our UCNPs implant to that of fiber optic, with both being implanted on different hemispheres. Two weeks post implantation; glial scarring was assessed in fixed brain slices by staining with reactive microglia (Iba+) as well as reactive astrocytes (GFAP+) antibodies respectively. Fiber optics evoked a significant amount of glial scarring consisted of reactive microglia and astrocytes. The UCNPs implant in contrast, showed significantly lower glial reactivity.

Several photosensitizers are already in the clinical use, including 5-ALA Gleolan, and Photofrin with an absorption wavelength of 630 nm. Importantly, a wide repertoire of core-shell UCNPs are available to cover the visible and NIR absorption spectra of photosensitizers. The design of the implant has been customized to meet 5-ALA absorption spectrum, allowing direct application to current clinical 5-ALA based photosensitizers, such as Gleolan and Levulan. 5-ALA is also critical in targeting dormant cancer cells, which together with PDT may confer management of tumor growth and recurrence of GBM. Apart from GBM, other forms of tumor beyond the brain are also known to uptake 5-ALA, which paves the way for potential PDT in the management of these tumors. As the implantable light delivery devices incorporating UCNPs are non-photo-bleaching, they can be chronically implanted in situ and be used as needed in the organ, without the need for battery replacement. The implantable light delivery devices incorporating UCNPs, allow retrieval from the tissue, when no longer needed, a feat not achievable with direct UCNPs suspension injection into tissue. The multi-spectra emission property of the implantable light delivery devices incorporating UCNPs is potentially expandable to activate multiple-drugs at once, without cross-interference. The simultaneous use of multiple PDT photosensitizers could confer a strategy to overcome resistance in tumor.

Furthermore, the implantable light delivery devices incorporating UCNPs are also applicable to other photo-based therapeutics beyond PDT, including wireless optogenetic study and photo-biomodulations.

Whilst the foregoing description has described exemplary embodiments, it will be understood by those skilled in the art that many variations of the embodiments can be made within the scope and spirit of the present invention.

1. An implantable light delivery device comprising:
a core portion cladded in a cladding, the core portion comprising up-conversion nano-particles encapsulated in an encapsulation material.

2. An implantable light delivery device according to claim 1 wherein the core portion and the cladding are flexible.

3. An implantable light delivery device according to claim 1, wherein a refractive index of the elongate core portion is greater than a refractive index of the cladding.

4. An implantable light delivery device according to claim 1, wherein the encapsulation material comprises hydrogel.

5. An implantable light delivery device according to claim 1, wherein the cladding forms a tube.

6. An implantable light delivery device according to claim 5, wherein at least one of end of the tube is closed by the cladding.

7. An implantable light delivery device according to claim 1, wherein the cladding comprises fluorinated ethylene propylene.

8. An implantable light delivery device according to claim 1, wherein the core portion is elongate.

9. An implantable light delivery device according to claim 1, wherein the UCNPs are selected to absorb near infra-red radiation having a wavelength in the range 700 nm to 1400 nm and to emit an emission spectrum in the having a wavelength in the visible range 300 nm to 700 nm in response to the near infra-red radiation.

10. An implantable light delivery device according to claim 8, wherein the excitation spectrum overlaps with an absorption spectrum of a photosensitizer.

11. An Implantable light delivery device according to any claim 1, wherein a concentration of the up-conversion nano-particles in the core portion is substantially uniform along the core portion.

12. An Implantable light delivery device according to claim 1, wherein a concentration of the up-conversion nano-particles in the core portion is varies along the core portion.

13. An implantable light delivery device according claim 1, comprising a plurality of elongate portions, each comprising a core portion cladded in a cladding, the core portion comprising up-conversion nano-particles encapsulated in an encapsulation material.

14. An implantable light delivery device according to claim 1, comprising a portion configured as a deformable cup or a deformable hollow sphere.

15. An implantable light delivery device according to claim 1, comprising an end portion configured as a solid sphere.

16. An implantable light delivery device according to claim 1, wherein the up-conversion nano-particles are configured to emit a first visible light emission in response to a first near infra-red radiation excitation and to emit a second visible light emission in response to a second near infra-red radiation excitation.

17. A photodynamic therapy method comprising:
implanting an implantable light delivery device comprising a core portion cladded in a cladding, the core portion comprising up-conversion nano-particles encapsulated in an encapsulation material;
administering a photosensitizer to the subject; and
illuminating the implantable light delivery device with radiation having excitation spectra corresponding to the up-conversion nano-particles.

18. A method of manufacturing an implantable light delivery device, the method comprising:
molding a mixture of an encapsulation material and up-conversion nano-particles;
causing the encapsulation material to polymerize.

19. A method according to claim 18, wherein molding the mixture comprises molding the mixture in a cladding.

20. A method according to claim 18, wherein causing the encapsulation material to polymerize comprises irradiating the encapsulation material with ultra-violet radiation.
